(19) United States
(12) Patent Application Publication
(10) Pub. No.: US 2007/0163368 A1
(43) Pub. Date: Jul. 19, 2007

(54) Defect detecting/degree-of-filling-in measuring apparatus for concrete during placement and method for defect detection/degree of filling-in measurement for the same

(76) Inventors: Hideyasu Yuki, Ota-ku (JP); Hiromi Nakagawa, Ota-ku (JP); Shigeki Seko, Inzai-shi (JP); Kenro Mitsui, Inzai-shi (JP); Souichi Kiya, Koto-ku (JP); Tadahiro Kakizawa, Inzai-shi (JP); Daisuke Saegusa, Inzai-shi (JP); Takeshi Suzuki, Inzai-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/653,263

(22) Filed: Jan. 16, 2007

(30) Foreign Application Priority Data
Jan. 17, 2006 (JP) 2006-008770

(51) Int. Cl. G01N 33/00

(52) U.S. Cl. 73/866

(57) ABSTRACT

The present invention provides a defect detecting/degree-of-filling-in measuring apparatus for concrete during placement that meets the tolerance for butting angle for the electrode with respect to the concrete form, and that is capable of carrying out measurement with the measurement depth being changed. The defect detecting/degree-of-filling-in measuring apparatus for concrete of the present invention comprises a detection section 42A for deeper-part measurement having a positive electrode 3 which surface to be butted against the concrete form is a curved surface and which is disposed in the middle portion of one end face of a cylindrical insulator, and a negative electrode 4 which is disposed at the other end face of the cylindrical insulator, being opposed to said positive electrode 3; and a detection section 42B for closer-part measurement having a spacing between the positive electrode 3 and the negative electrode 4 that is smaller than that for detection section 42A for deeper-part measurement at both ends of a gripper 41; determines the electrostatic capacitance corresponding to the deeper-part and the closer-part of the concrete during placement by means of the deeper-part measuring circuit and the closer-part measuring circuit; determines the degree of filling-in for the deeper part and the closer part for the object under measurement by means of the arithmetic circuit; further determines whether the moisture content and the concrete filled-in quantity for the concrete during placement are adequate; and displays the result in the display section 46.

### BACKGROUND OF THE INVENTION

1. Field of the Invention

The present invention relates to a defect detection apparatus for concrete during placement, and particularly relates to a defect detecting/degree-of-filling-in measuring apparatus for concrete during placement that is capable of simply detecting the occurrence of a defect of the concrete during placement from outside of a concrete form, and a method for defect detection/degree of filling-in measurement for the same.

2. Description of the Related Art

The qualities, such as the strength and the appearance, of the concrete during placement on the construction site, and the like depends upon the degree of filling in.

When the concrete is placed in a required portion to construct a building, or the like, inadequate filling in of the concrete after placement makes it impossible to construct a building, or the like, which has a good appearance, and for which the quality can be assured over a long term. Therefore, on the construction site, and the like, where concrete placement is carried out, it is necessary to quickly detect a defect of inadequate degree of filling in.

The quality of the building material, such as concrete during placement, depends upon the moisture content thereof, thus in case where concrete is placed in a necessary portion to construct a building, or the like, too high or too low a moisture content of the concrete after placement will make it impossible to adequately perform the finishing for constructing a high-quality building, or the like. Therefore, on the concrete construction site, and the like, it is required to quickly detect a defect of inadequate moisture content, or the like. From the viewpoint of this, a moisture content meter of type which is butted against object under measurement from the surface thereof has been conventionally available.

As compared to the type which is embedded in the inside of the object under measurement, and that which sandwiches the object under measurement from both sides, the above-mentioned moisture content meter of type which is butted against object under measurement from the surface thereof is capable of making operation, simply if the measurement surface is exposed, thus it is more practical on the concrete construction site, for example.

In addition, when a conventional moisture content meter is used in the above-mentioned case on the construction site, or the like, it may be necessary to know not only the moisture content of the whole, but also that of each portion. Therefore, it may be required to make approximate determination of the moisture content distribution along the depth of the object under measurement.

By the way, a high-frequency moisture content meter has been conventionally used for measurement of the moisture content of objects under measurement. With this, the electrostatic capacitance, which varies depending upon the change in moisture content, is determined by measuring the high-frequency current flowing through the object under measurement to calculate the moisture content.

As shown in FIG. 21 and FIG. 22, the conventional moisture content meter 100 is configured such that two bar-like electrodes 102, 103 having a length “b” and supported in a parallel arrangement by an insulator 101 with a spacing “a” are butted against the measurement surface 111 of an object under measurement 110, and the electrostatic capacitance across both electrodes 102, 103 is measured for computation of the moisture content of the object under measurement 110.

However, with such a conventional electrode structure, in case where the measurement surface 111 is not flat, a part of the electrode 102, 103 is lifted from (brought into non-contact with) the measurement surface 111. As a result of this, the flow of the high-frequency current from the negative electrode 102, 103 to the measurement surface 111 is hindered, resulting in occurrence of a measurement error.

With the conventional electrode structure, if the spacing “a” between the electrodes 102, 103 is reduced, the lifting is made difficult to be caused, however, because the spacing “a” is small, the change in electrostatic capacitance for a given moisture content is small, which causes the measurement to be influenced by the instability of the measuring circuit, making it impossible to carry out stable measurement.

Next, let's consider the wooden plate as the object under measurement 110. The measurement which is carried out with the negative electrodes 102, 103 being butted against the wooden plate as the object under measurement 110 in parallel with the moire thereof as shown in FIG. 23, and the measurement which is carried out with the electrodes being butted against the wooden plate at right angles to the moire as shown in FIG. 24 provide different measured values of electrostatic capacitance, even if the same area is measured. Therefore, with the electrode structure of the moisture content meter 100 as shown in FIG. 21, there occurs a problem that the measurement result can vary, depending upon the butting angle.

Further, with the moisture content meter 100 as shown in FIG. 21, it has a structure in which the electrodes 102, 103 are mounted to one surface of the insulator 101 in a parallel arrangement, thus the measurement range is uniform, and it is impossible to carry out measurement with the measurement depth being changed along the direction of depth of the object under measurement 110.

In the patent literature 1, a method for testing of concrete quality which is configured such that, by inserting the detection device equipped with an electrode plate for measuring the electrostatic capacitance for the mortar into the area where the concrete is placed in the structure, the electrostatic capacitance for the concrete is measured to determine whether the unit moisture content of the concrete to be placed is adequate, and a detection device for the same have been proposed.

However, the method for testing of concrete quality as disclosed in this patent literature 1 also provides a uniform measurement range, and is not configured such that the measurement depth for the concrete during placement is changed in conducting the quality test.

[Patent literature 1] Japanese Patent Publication No. JP/2004-077454A

### SUMMARY OF THE INVENTION

Then, the purpose of the present invention is to solve the above-mentioned problems, and to provide a defect detecting/degree-of-filling-in measuring apparatus and a method for defect detection/degree of filling-in measurement for concrete during placement which meet the tolerance for butting angle for the electrode with respect to the concrete form when the electrodes are butted against the concrete form for detection of the occurrence of a defect of the concrete during placement, and which have a novel configuration allowing measurement with the measurement depth being changed have not been conventionally available.

An aspect of the present invention provides a defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising a detection section having one electrode which surface to be butted against the concrete form is a curved surface; an insulator which is disposed such that a part of one end face thereof is adjacent to this one electrode; and the other electrode which is disposed at the other end face of said insulator, being opposed to said one electrode.

According to the present invention, the following effects will be provided.

According to the inventions as stated in claim 1 and claim 2, the defect detecting/degree-of-filling-in measuring apparatus for concrete during placement is configured such that it comprises a detection section having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode; from a detection signal of the detection section, the electrostatic capacitance across said positive electrode and negative electrode is determined; and further, the degree of filling-in for the concrete during placement is determined to be displayed, thus with the tolerance for butting angle for the positive electrode with respect to the concrete form being met, the degree of filling-in for the concrete during placement can be accurately measured for precise detection of the occurrence of a defect.

According to the inventions as stated in claim 3 and claim 4, with the same configuration as that for the invention as stated in claim 1 and a configuration in which the detection section for deeper-part measurement and that for closer-part measurement which have different spacings between electrodes are provided at both ends of the gripper, respectively, the respective degrees of filling-in for the deeper part and the closer part of the concrete during placement can be accurately measured from the detection signals of these detection sections for precise detection of the occurrence of a defect, with the tolerance for butting angle for the positive electrode with respect to the concrete form being met. In addition, according to the invention as stated in claim 5, the same effects as those by the inventions as stated in claim 3 and claim 4 can be provided with the configuration in which the central portion of the gripper is bent at 90 degrees.

According to the inventions as stated in claim 6 and claim 7, with the same configuration as that for the invention as stated in claim 1, and a configuration in which the detection section for deeper-part measurement and that for closer-part measurement which have different spacings between electrodes are provided as parts bifurcated from the gripper, the respective degrees of filling-in for the deeper part and the closer part of the concrete during placement can be accurately measured from the detection signals of these detection sections for precise detection of the occurrence of a defect, with the tolerance for butting angle for the positive electrode with respect to the concrete form being met.

According to the inventions as stated in claim 8 and claim 9, with the same configuration as that for the invention as stated in claim 1, and a configuration in which the detection section is provided with one positive electrode and two negative electrodes having large and small different distances from the positive electrode, the respective degrees of filling-in for the deeper part and the closer part of the concrete during placement can be accurately measured for precise detection of the occurrence of a defect, with the tolerance for butting angle for the positive electrode with respect to the concrete form being met.

According to the inventions as stated in claim 10 and claim 11, with a configuration in which, with respect to the gripper provided with the same detection section for closer-part measurement as that for the invention as stated in claim 1, the detection section for deeper-part measurement is removably disposed, the respective degrees of filling-in for the deeper part and the closer part of the concrete during placement can be accurately measured for precise detection of the occurrence of a defect, with the tolerance for butting angle for the positive electrode with respect to the concrete form being met.

According to the inventions as stated in claim 12 and claim 13, with a configuration in which the detection section has the negative electrode which is disposed such that the spacing between it and the positive electrode is adjustable, the respective degrees of filling-in for the deeper part and the closer part of the concrete during placement can be accurately measured for precise detection of the occurrence of a defect, with the tolerance for butting angle for the positive electrode with respect to the concrete form being met.

According to the invention as stated in claim 14, the defect detection/degree of filling-in measurement method for concrete during placement provides a defect detection/degree of filling-in measurement method for concrete during placement which uses any one of the defect detecting/degree-of-filling-in measuring apparatuses for concrete during placement as stated in claim 3 to claim 13, wherein the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect, thus it allows any one of the defect detecting/degree-of-filling-in measuring apparatuses for concrete during placement as stated in claim 3 to claim 13 to be used for easily detecting the filling-in defect of the concrete during placement.

### BRIEF DESCRIPTION OF THE DRAWINGS

FIG. 1 is an explanatory drawing showing the configuration of the measurement part of a defect detecting/degree-of-filling-in measuring apparatus of an embodiment 1 of the present invention;

FIG. 2 is a plan view of the measurement part of the defect detecting/degree-of-filling-in measuring apparatus of the present invention when viewed from the positive electrode side;

FIG. 3 is a table giving the correlation between the inclination angle for the defect detecting/degree-of-filling-in measuring apparatus of the present embodiment 1 and the concrete filling-in degree;

FIG. 4 is a schematic sectional view illustrating a specific example of configuration of the measurement part of the defect detecting/degree-of-filling-in measuring apparatus of the present embodiment 1;

FIG. 5 is a schematic view illustrating the state in which the measurement part of the defect detecting/degree-of-filling-in measuring apparatus of the present embodiment 1 is inclined;

FIG. 6 is schematic sectional view illustrating a specific example of configuration of the defect detecting/degree-of-filling-in measuring apparatus of the present embodiment 1;

FIG. 7 is a block diagram illustrating the circuitry of the defect detecting/degree-of-filling-in measuring apparatus of the present embodiment 1;

FIG. 8 is a front view of a defect detecting/degree-of-filling-in measuring apparatus of an embodiment 2 of the present invention;

FIG. 9 is a block diagram illustrating the circuitry of the defect detecting/degree-of-filling-in measuring apparatus of the present embodiment 2;

FIG. 10 is a front view illustrating an modification of the defect detecting/degree-of-filling-in measuring apparatus of the embodiment 2 of the present invention;

FIG. 11 is a front view illustrating another modification of the defect detecting/degree-of-filling-in measuring apparatus of the embodiment 2;

FIG. 12 is a sectional view illustrating the measurement part of an embodiment 3 of the present invention;

FIG. 13 is a block diagram illustrating the circuitry of the present embodiment 3;

FIG. 14 is a front view illustrating the measurement part of an embodiment 4 of the present invention;

FIG. 15 is a front view illustrating the measurement part of the present embodiment 4;

FIG. 16 is a front view illustrating the measurement part for closer-part measurement of the present embodiment 4;

FIG. 17 is a sectional view illustrating the measurement part (at the time of deeper-part measurement) of an embodiment 5 of the present invention;

FIG. 18 is a sectional view illustrating the measurement part (at the time of closer-part measurement) of an embodiment 5 of the present invention;

FIG. 19 is a regression analysis diagram about concrete and (void+rock pocket) on the basis of the measured values obtained using the defect detecting/degree-of-filling-in measuring apparatus of the embodiment 1 of the present invention;

FIG. 20 is a bar graph illustrating the relationship between the determination score about concrete and (void+rock pocket) and the frequency, which has been prepared on the basis of a regression analysis result as shown FIG. 19;

FIG. 21 is a front view illustrating the measurement part of a conventional moisture content meter;

FIG. 22 is a bottom view illustrating the measurement part of the conventional moisture content meter;

FIG. 23 is an explanatory drawing illustrating the state in which the measurement part of the conventional moisture content meter is disposed in parallel with the moire of a wooden plate;

FIG. 24 is an explanatory drawing illustrating the state in which the measurement part of the conventional moisture content meter is disposed at right angles to the moire of a wooden plate.

### BEST MODE FOR CARRYING OUT THE INVENTION

The present invention has achieved the purpose of providing a defect detecting/degree-of-filling-in measuring apparatus for concrete during placement that meets the tolerance for butting angle for the electrode with respect to the concrete form, and that is capable of carrying out measurement with the measurement depth being changed, by taking a configuration which provides a defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising a detection section for deeper-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode; and a detection section for closer-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode, the spacing between said positive electrode and negative electrode being smaller than that for the detection section for deeper-part measurement; at both ends of a gripper, and having a deeper-part measuring circuit which determines the electrostatic capacitance across said positive electrode and negative electrode from a detection signal of said detection section for deeper-part measurement; a closer-part measuring circuit which determines the electrostatic capacitance across said positive electrode and negative electrode from a detection signal of said detection section for closer-part measurement; an arithmetic circuit which processes the respective measured signals of said deeper-part measuring circuit and closer-part measuring circuit through the switching action to determine whether the degree of filling-in for said concrete during placement is adequate; and a display section which displays the result of operation of the arithmetic circuit.

### EMBODIMENTS

Hereinbelow, embodiments of the present invention will be described in detail.

### Embodiment 1

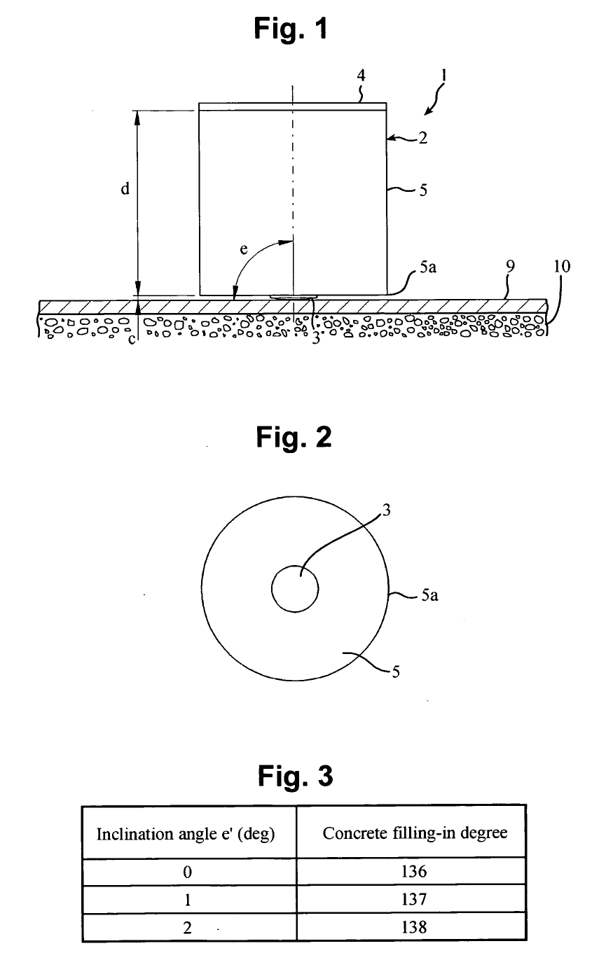

With reference to FIG. 1 to FIG. 7, a defect detecting/degree-of-filling-in measuring apparatus for concrete during placement of an embodiment 1 of the present invention will be described.

First of all, with reference to FIG. 1 to FIG. 3, a theoretical description of the present invention will be given.

As shown in FIG. 1 and FIG. 2, if a detection section 2 of a defect detecting/degree-of-filling-in measuring apparatus 1 for concrete during placement (hereinafter, to be called a “defect detecting/degree-of-filling-in measuring apparatus”) has a structure wherein a positive electrode 3 which end face is formed in the shape of a curved surface is disposed at one end of a cylindrical insulator 5 (with a dimension “d”), while a negative electrode 4 is at the other end, the electric field across the positive electrode 3 and the negative electrode 4 is distributed through a concrete during placement 10 which is an object under measurement existing with a concrete form 9 made up of such as a wood plate, or the like, being provided between it and the positive electrode 3, thus the dielectric constant therefor will have a great effect on the electrostatic capacitance across the positive electrode 3 and the negative electrode 4.

FIG. 1 illustrates the arrangement of the defect detecting/degree-of-filling-in measuring apparatus 1 and the concrete form 9 when a floor body is constituted, however, the positional relationship between the defect detecting/degree-of-filling-in measuring apparatus 1 and the concrete form 9 may, of course, vary depending upon the disposition angle for the concrete form 9 in the concrete placement portion.

The dielectric constant for the concrete during placement 10 is dependent upon the moisture content, thus by measuring the electrostatic capacitance across the positive electrode 3 and the negative electrode 4, it is made possible to carry out the moisture content detection, the concrete filled-in quantity detection, and the defect determination for the pertinent concrete during placement 10.

In said detection section 2, if, as compared to the dimension of protrusion, “c”, of the positive electrode 3 from the one end of the insulator 5, the degree of surface irregularities of the concrete form 9 is small, the positive electrode 3 will not be lifted from the surface of the concrete form 9, resulting in the contact with the measurement surface being positively maintained, which allows stable moisture content measurement and concrete filled-in quantity measurement to be performed.

In addition, the angle deviation “e” of the perpendicular direction for the detection section 2 is considered in such a way that, if the inclination angle “e′” of the positive electrode 3 from the horizontal surface (see FIG. 5) when the detection section 2 is inclined of the perpendicular direction is relatively small (as small as 1 or 2 deg), the effect of the inclination angle “e′” on the measured value for concrete filling-in degree is in the range of 0 to 2 deg as shown in FIG. 3, which is of an almost negligible degree.

This inclination angle “e′”, i.e., said deviation “e” will be held to within such a small angular range, even if the measurement personnel roughly determines that the detection section 2 is substantially perpendicular (at approx. 90 degrees) with respect to the measurement surface.

Further, if the circumferential edge part 5a of the insulator 5 is butted against the concrete form 9, it is sensed by the measurement personnel with the feeling to know that the inclination is too much.

As can be understood from these, if the measurement personnel has a normal attentiveness, it is avoidable that, in measuring the moisture content and the concrete filled-in quantity for the concrete during placement 10, the butting angle for the positive electrode 3 is increased, resulting in the tolerance for measurement error being not met. However, the tolerance for butting angle changes depending upon the tolerance for measurement error. It must be noted that, if the tolerance for measurement error is loose, the tolerance for butting angle may be loose accordingly.

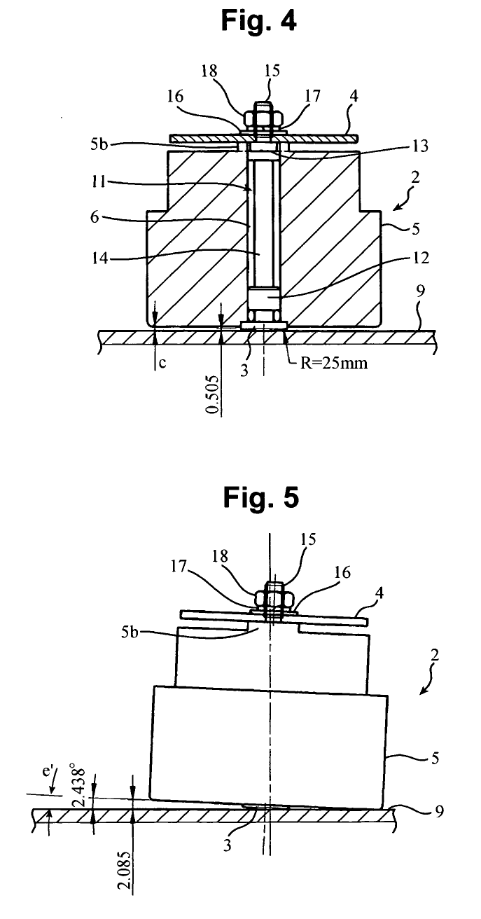

Next, with reference to FIG. 4 and FIG. 5, an example of specific configuration of a detection section 2 of the present embodiment 1 and the inclination angle “e′” will be described.

The detection section 2 of the defect detecting/degree-of-filling-in measuring apparatus 1 as shown in FIG. 4 has a structure wherein, at one end of the cylindrical and shouldered insulator 5, the positive electrode 3 in the shape of a small diameter disk which middle portion has a thickness of 2 mm or so and which end face has a curved surface with a radius R=25 mm is disposed, while, at the other end of the insulator 5, the negative electrode 4 in the shape of a disk is disposed.

Said positive electrode 3 and negative electrode 4 are connected to each other in the insulated state and like an integral part by a connection member 11 disposed in a through-hole 6 provided in the middle portion of the insulator 5. In other words, said connection member 11 connects between a lower connection receiving part 12 anchored to the positive electrode 3 and an upper connection receiving part 13 from which a screw part 15 is protruded to be fitted into the middle portion of the negative electrode 4, by means of a connecting rod 14 disposed in the through-hole 6; further the middle portion of the negative electrode 4 is fitted to the screw part 15; the flat washer 16 and the spring washer 17 are fitted to the screw part 15; and a nut 18 is tightened, whereby said positive electrode 3 and negative electrode 4 are disposed, being fixed such that they are opposed to each other, sandwiching the insulator 5.

Said lower connection receiving part 12, connecting rod 14, and upper connection receiving part 13 are formed of an insulating material such as a synthetic resin, or the like.

In FIG. 4, a reference numeral of 5b indicates a protrusion provided on the insulator 5 to receive the negative electrode 4.

With the detection section 2 of the defect detecting/degree-of-filling-in measuring apparatus 1 as shown in FIG. 4, the dimension of protrusion, “c”, from the one end of the insulator 5 of the positive electrode 3 is set at, for example, 1 mm, and the dimension from the point where the curved surface is started at the side surface of the positive electrode 3 to the surface of the concrete form 9 is set at 0.505 mm, whereby, as shown in FIG. 5, when the detection section 2 is inclined such that the circumferential edge part 5a thereof is contacted with the concrete form 9, the inclination angle “e′” will be equal to 2.438 deg, and the maximum distance between the detection section 2 and the surface of the concrete form 9 will be 2.085 mm.

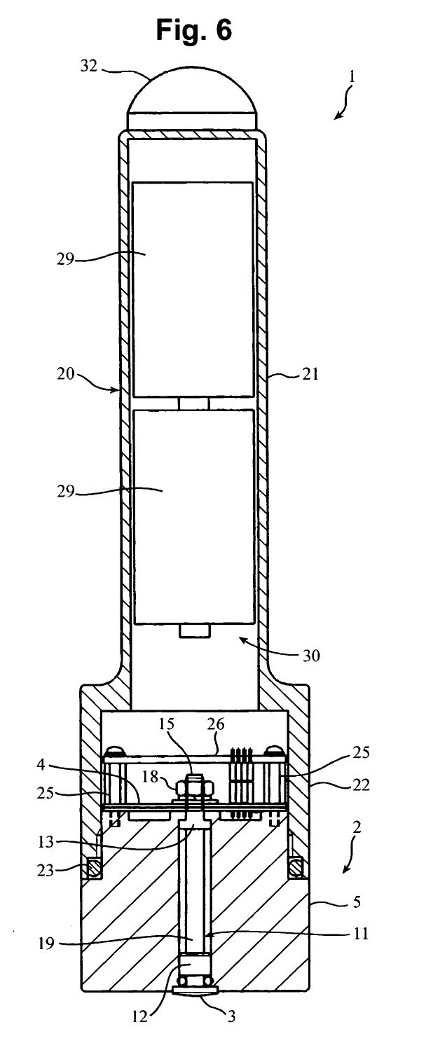

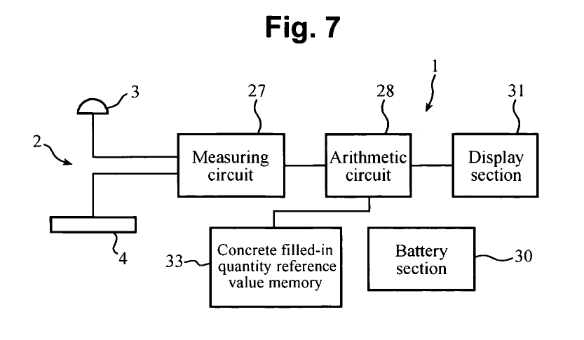

Next, with reference to FIG. 6 and FIG. 7, an example of specific configuration of the defect detecting/degree-of-filling-in measuring apparatus 1 of the present embodiment 1 will be described.

The defect detecting/degree-of-filling-in measuring apparatus 1 of the present embodiment 1 comprises a shouldered cylindrical gripper 20 which is made up of a smaller diameter cylindrical part 21 for holding that is formed of an insulating material, and a larger diameter cylindrical part 22 for accommodating the detection section 2 as shown in FIG. 4, and is configured such that, into the opening side of the larger diameter cylindrical part 22, the portion of the negative electrode of the insulator 5 is fitted, with an O-ring 23 being provided between the larger diameter cylindrical part 22 and the outer edge of the insulator 5 in said detection section 2, for installing the negative electrode 4 inside said opening.

In addition, to the negative electrode 4 inside said opening, a printed wiring board 26 loaded with a measuring circuit 27 (for example, a high-frequency bridge circuit, or the like), an arithmetic circuit 28, and the like, through a spacer part 25 is mounted. Further, inside said smaller diameter cylindrical part 21, a battery section 30 which connects in series two dry batteries 29 made up of, for example, a D-type, C-type, or the like is accommodated.

FIG. 7 illustrates a circuitry of the defect detecting/degree-of-filling-in measuring apparatus 1 of the present embodiment 1, which is configured such that said positive electrode 3 and the negative electrode 4 are connected to a measuring circuit 27 for measuring the electrostatic capacitance across the positive electrode 3 and the negative electrode 4; the measured value for the measuring circuit 27 is computed by an arithmetic circuit 28 for determining the degree of filling-in for the concrete during placement 10; further the data for the preset moisture content reference value and concrete filled-in quantity reference value for the concrete during placement 10 is read out from a moisture content reference value memory (a concrete filled-in quantity reference value memory) 33 to be compared with said degree of filling-in determined; whether the moisture content and the concrete filled-in quantity for the concrete during placement 10 are adequate is determined; and a display section 31 constituted by a liquid crystal display, or the like, displays said degree of filling-in, and the result of adequacy determination (for example, filling-in being too less, filling-in being less, filling-in being adequate, or the like). In FIG. 6, showing the display section 31 is abbreviated.

In addition, in said arithmetic circuit 28, the relationship among the output value from said measuring circuit 27, the degree of filling-in, the moisture content reference value, and the concrete filled-in quantity reference value is programmed for carrying out the above-mentioned arithmetic operation. In FIG. 6, a reference numeral of 32 indicates an end member which is formed in the shape of a curved surface.

According to the defect detecting/degree-of-filling-in measuring apparatus 1 of the present embodiment 1, the detection section 2 which has a configuration wherein, at one end of the insulator 5, the positive electrode 3 which end face is in the shape of a curved surface is disposed, while, at the other end thereof, the negative electrode 4 is disposed is used, and the positive electrode 3 of this detection section 2 is butted against the concrete form 9, whereby, even if the defect detecting/degree-of-filling-in measuring apparatus 1 is inclined of the perpendicular direction, the degree of filling-in for the concrete during placement 10 is accurately measured, with the inclination angle “e′” being held to a minimum, which allows the presence of a defect to be reliably detected.

### Embodiment 2

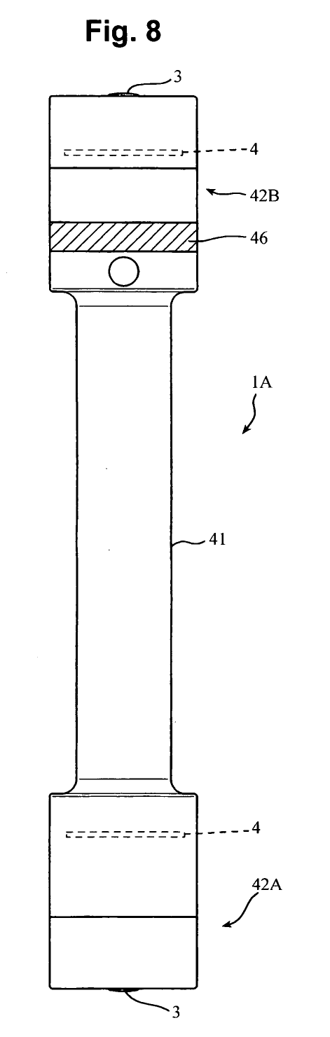

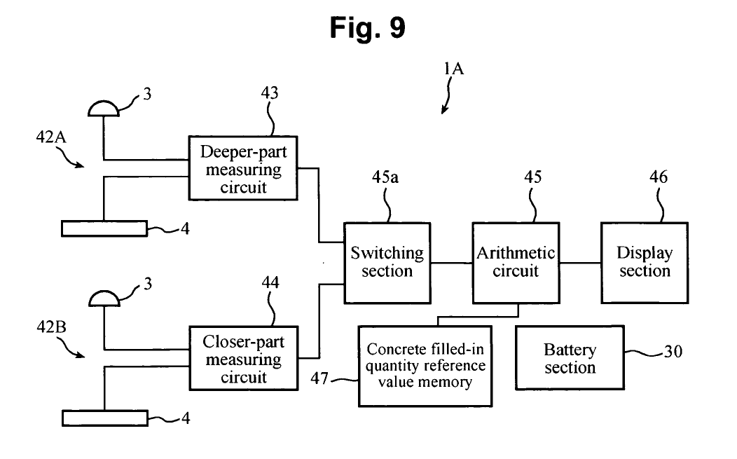

Next, with reference to FIG. 8 and FIG. 9, an embodiment 2 of the present invention will be described.

A defect detecting/degree-of-filling-in measuring apparatus 1A of the present embodiment 2 as shown in FIG. 8 and FIG. 9 features that, at both ends of a single cylindrical gripper 41, a detection section 42A for deeper-part measurement of the concrete during placement 10 and a detection section 42B for closer-part measurement thereof are mounted.

Herein, for the following description, it is premised that the term “deeper part” refers to a portion of the concrete during placement 10 that is given at a relatively large distance from the positive electrode 3 along the direction of depth, while the term “closer part” refers to a portion of the concrete during placement 10 that is given at a relatively small distance from the positive electrode 3 along the direction of depth.

Said detection section 42A and detection section 42B have the same configuration as that of the detection section 2 as shown in FIG. 4, and the spacing between the positive electrode 3 and the negative electrode 4 (indicated with a dotted line in FIG. 8) and the thickness of the insulator 5 are larger for the detection section 42A, while they are smaller for the detection section 42B.

In addition, as shown in FIG. 9, the defect detecting/degree-of-filling-in measuring apparatus 1A of the present embodiment 2 is configured such that the positive electrode 3 and the negative electrode 4 of said detection section 42A are connected to the deeper-part measuring circuit 43 for measuring the electrostatic capacitance across the positive electrode 3 and the negative electrode 4; the measured value for the deeper-part measuring circuit 43 is inputted to the arithmetic circuit 45 by the switching of the switching section 45a; the arithmetic circuit 45 performs computation to determine the degree of filling-in for the deeper part of the concrete during placement 10; further the data for the preset moisture content reference value and concrete filled-in quantity reference value for the concrete during placement 10 is read out from a moisture content reference value memory (a concrete filled-in quantity reference value memory) 47 to be compared with said degree of filling-in determined; whether the moisture content and the concrete filled-in quantity for the concrete during placement 10 are adequate is determined; and a display section 46 constituted by a liquid crystal display, or the like, displays said degree of filling-in, and the result of adequacy determination (for example, filling-in being too less, filling-in being less, filling-in being adequate, or the like).

In addition, the defect detecting/degree-of-filling-in measuring apparatus 1A of the present embodiment 2 is configured such that the positive electrode 3 and the negative electrode 4 of said detection section 42B are connected to the closer-part measuring circuit 44 for measuring the electrostatic capacitance across the positive electrode 3 and the negative electrode 4; the measured value for the closer-part measuring circuit 44 is inputted to the arithmetic circuit 45 by the switching of the switching section 45a; the arithmetic circuit 45 performs computation to determine the degree of filling-in for the deeper part of the concrete during placement 10; further the data for the preset moisture content reference value and concrete filled-in quantity reference value for the concrete during placement 10 is read out from a moisture content reference value memory (a concrete filled-in quantity reference value memory) 47 to be compared with said degree of filling-in determined; whether the moisture content and the concrete filled-in quantity for the concrete during placement 10 are adequate is determined; and a display section 46 constituted by a liquid crystal display, or the like, displays said degree of filling-in, and the result of adequacy determination (for example, filling-in being too less, filling-in being less, filling-in being adequate, or the like).

According to the defect detecting/degree-of-filling-in measuring apparatus 1A of the present embodiment 2, the detection section 42A for deeper-part measurement of the concrete during placement 10 and the detection section 42B for closer-part measurement thereof are provided at both ends of a single cylindrical gripper 41, thus the moisture content measurement (the concrete filled-in quantity measurement) of the deeper part and the defect detection thereof by butting the positive electrode 3 of the detection section 42A for deeper-part measurement against the concrete form 9, and the moisture content measurement (the concrete filled-in quantity measurement) of the closer part and the defect detection thereof by regripping the gripper and butting the positive electrode 3 of the detection section 42B for closer-part measurement against the concrete form 9 can be selectively and simply carried out by means of a single defect detecting/degree-of-filling-in measuring apparatus 1A.

In this case, assuming that the measured value when the detection section 42A for deeper-part measurement is used is “A”, and the measured value when the detection section 42B for closer-part measurement is used is “B”, the value “A−B” corresponds to the degree of filling-in for the deeper part, and the value “B” to the degree of filling-in for the closer one. However, this provides an example, and adoption of some other type of computation method may give a higher accuracy, the adequate type of computation method varying depending upon the region where the moisture content is to be measured, that where the concrete filled-in quantity is to be measured, the material composition for the concrete during placement 10, and the like.

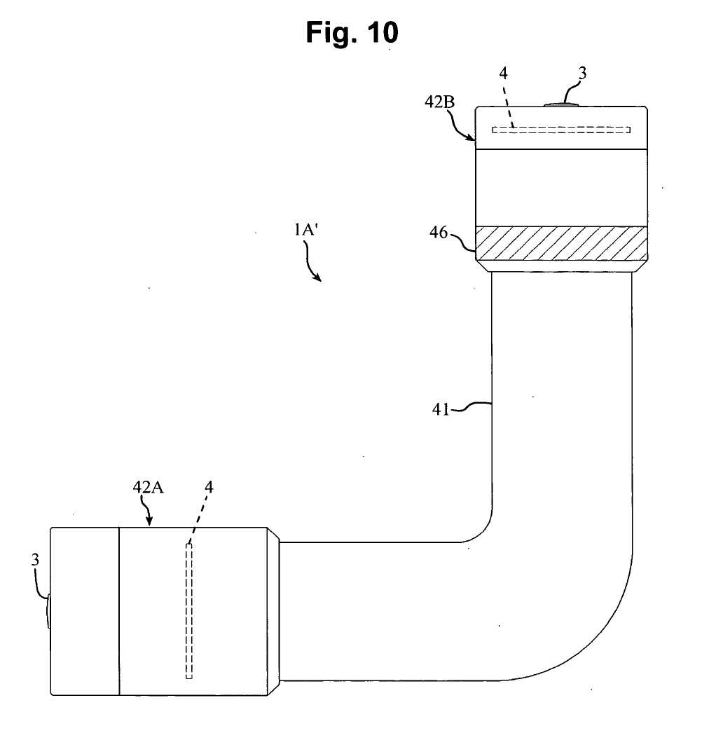

FIG. 10 illustrates a defect detecting/degree-of-filling-in measuring apparatus 1A′, which is a modification of the defect detecting/degree-of-filling-in measuring apparatus 1A of the present embodiment 2, and has the same basic configuration as that of said defect detecting/degree-of-filling-in measuring apparatus 1A, except that the central portion of the gripper 41 is bent at 90 degrees, allowing the moisture content measurement (the concrete filled-in quantity measurement) of the deeper part and the defect detection thereof by butting the positive electrode 3 of the detection section 42A for deeper-part measurement against the concrete form 9, and the moisture content measurement (the concrete filled-in quantity measurement) of the closer part and the defect detection thereof by regripping the gripper and butting the positive electrode 3 of the detection section 42B for closer-part measurement against the concrete form 9 to be selectively and simply carried out by means of a single defect detecting/degree-of-filling-in measuring apparatus 1A as with said defect detecting/degree-of-filling-in measuring apparatus 1A.

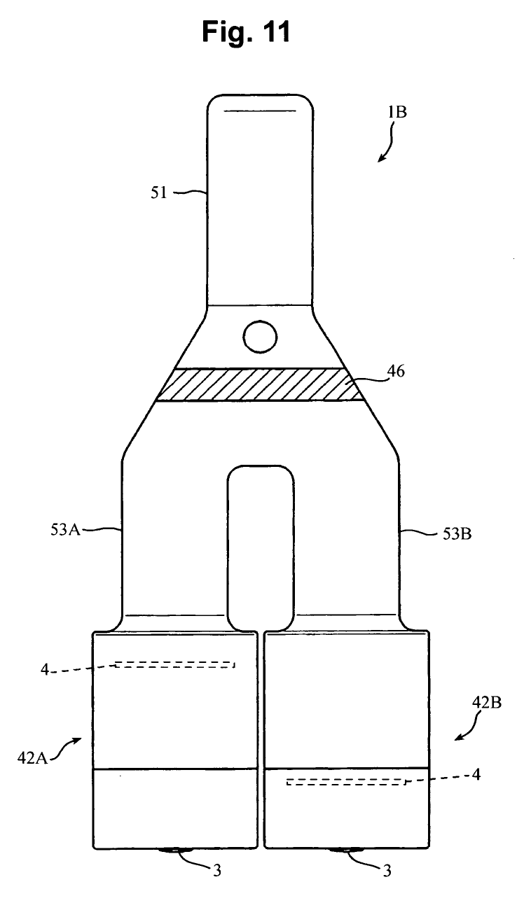

FIG. 11 illustrates another modification of the embodiment 2 of the present invention, and this defect detecting/degree-of-filling-in measuring apparatus 1B as a modification features that detection section holding parts 53A, 53B, which are bifurcated from the gripping part 51, are equipped with a detection section 42A for deeper-part measurement for the concrete during placement 10 and a detection section 42B for closer-part measurement therefor, respectively.

Said detection section 42A and detection section 42B have the same configuration as that of said detection section 2 as shown in FIG. 4, and the spacing between the positive electrode 3 and the negative electrode 4 (indicated with a dotted line in FIG. 11) and the thickness of the insulator 5 are larger for the detection section 42A, while they are smaller for the detection section 42B.

With this defect detecting/degree-of-filling-in measuring apparatus 1B as a modification, the same circuitry as that as shown in FIG. 9 is adopted, and the system is configured such that the arithmetic circuit 45 is switched to be operated either for moisture content measurement and concrete filled-in quantity measurement for the deeper part with the positive electrode 3 of the detection section 42A for deeper-part measurement being butted against the concrete form 9, and for moisture content measurement and concrete filled-in quantity measurement for the closer part with the positive electrode 3 of the detection section 42B for closer-part measurement being butted against the concrete form 9.

According to this defect detecting/degree-of-filling-in measuring apparatus 1B as a modification, a single defect detecting/degree-of-filling-in measuring apparatus 1B having a configuration of bifurcation is capable of carry outing either the moisture content measurement and concrete filled-in quantity measurement for the deeper-part with the positive electrode 3 of the detection section 42A for deeper-part measurement being butted against the concrete form 9, or the moisture content measurement and concrete filled-in quantity measurement for the closer-part with the positive electrode 3 of the detection section 42B for closer-part measurement being butted against the concrete form 9, through the switching action.

### Embodiment 3

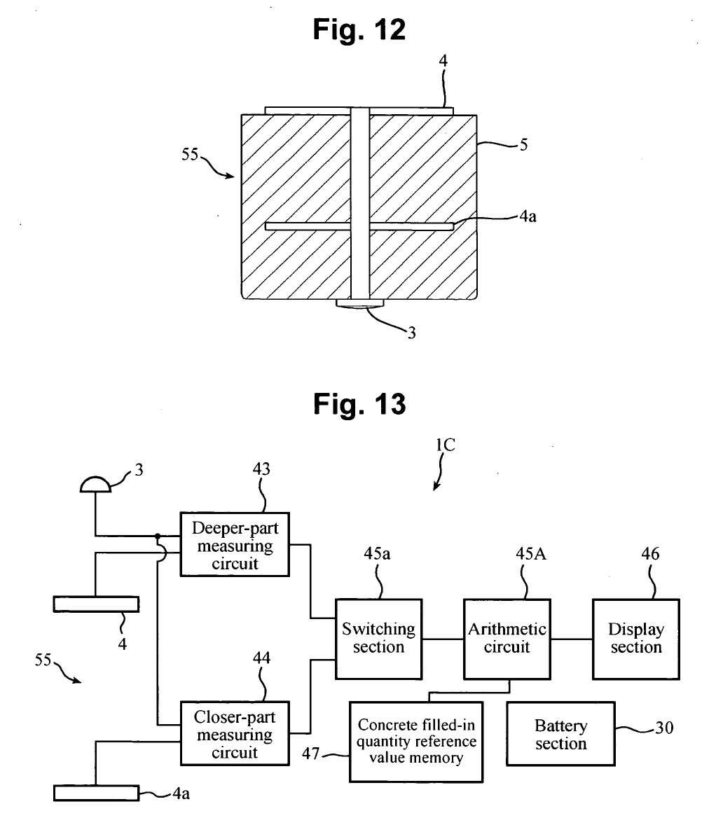

Next, with reference to FIG. 12 and FIG. 13, a detection section 55 in an embodiment 3 of the present invention will be described. The detection section 55 of the present embodiment 3 features that it has a configuration wherein, at one end of an insulator 5, a positive electrode 3 is disposed, and at the other end thereof, a negative electrode 4 is disposed, with another negative electrode 4a being disposed inside of the insulator 5 in parallel with the negative electrode 4, thus two negative electrodes 4,4a being provided.

In other words, the detection section 55 is configured such that it uses the positive electrode 3 and the negative electrode 4 to carry out moisture content measurement and concrete filled-in quantity measurement for the deeper part of a concrete during placement 10, or uses the positive electrode 3 and the negative electrode 4a to carry out moisture content measurement and concrete filled-in quantity measurement for the closer part of a concrete during placement 10 through the switching action.

FIG. 13 shows a circuitry for a defect detecting/degree-of-filling-in measuring apparatus 1C including the detection section 55 of the present embodiment 3. This circuitry is configured such that the positive electrode 3 and the negative electrode 4 are connected to a deeper-part measuring circuit 43 of said detection section 55 to measure the electrostatic capacitance across the positive electrode 3 and the negative electrode 4; the measured value for the deeper-part measuring circuit 43 is fed to an arithmetic circuit 45A through the action of a switching section 45a; the arithmetic circuit 45A performs computation to determine the degree of filling-in for the deeper part of the concrete during placement 10; in addition, the positive electrode 3 and the negative electrode 4a are connected to a closer-part measuring circuit 44 of said detection section 55 to measure the electrostatic capacitance across the positive electrode 3 and the negative electrode 4a; the measured values for the deeper-part measuring circuit 43 and the closer-part measuring circuit 44 are fed to the arithmetic circuit 45A through the action of the switching section 45a; the arithmetic circuit 45A performs computation to determine the respective degrees of filling-in for the deeper part and the closer part of the concrete during placement 10; further the data for the preset moisture content reference value and concrete filled-in quantity reference value for the concrete during placement 10 is read out from a moisture content reference value memory (a concrete filled-in quantity reference value memory) 47 to be compared with said respective degrees of filling-in determined for the deeper part and the closer part; whether the moisture content and the concrete filled-in quantity for the concrete during placement 10 are adequate is determined for each of the deeper part and the closer part; and a display section 46 constituted by a liquid crystal display, or the like, displays said degree of filling-in, and the result of adequacy determination (for example, filling-in being less, filling-in being adequate, filling-in being too much, or the like).

According to the defect detecting/degree-of-filling-in measuring apparatus 1C of the present embodiment 3, the detection section 55 having one positive electrode 3 and two negative electrodes 4, 4a is provided, thus a single defect detecting/degree-of-filling-in measuring apparatus 1C having a simple configuration is capable of carrying out moisture content measurement, concrete filled-in quantity measurement, and defect detection for the deeper part of the concrete during placement 10, or moisture content measurement, concrete filled-in quantity measurement, and defect detection for the closer part of the concrete during placement 10 through the switching action.

### Embodiment 4

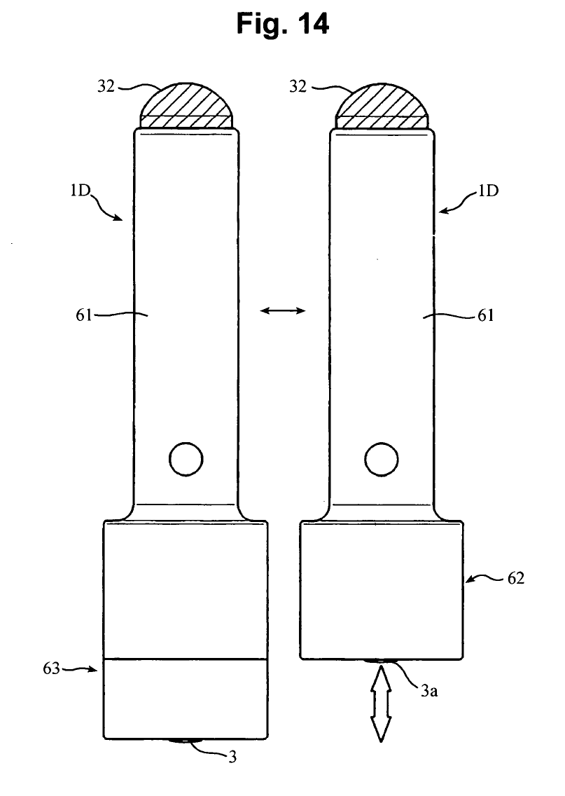

Next, with reference to FIG. 14 to FIG. 16, a defect detecting/degree-of-filling-in measuring apparatus 1D of an embodiment 4 of the present invention will be described.

The defect detecting/degree-of-filling-in measuring apparatus 1D of the present embodiment 4 has the same basic configuration as that of the defect detecting/degree-of-filling-in measuring apparatus 1 for concrete during placement of the embodiment 1 as shown in FIG. 6, except that it features that, at one end of a cylindrical gripper 61, a closer-part measurement part 62 which has a positive electrode 3 for closer-part measurement at the end face thereof and incorporates a negative electrode 4 inside thereof is provided, and a deeper-part measurement part 63 which has a positive electrode 3 for deeper-part measurement at the end face thereof is removably provided as an attachment to the closer-part measurement part 62.

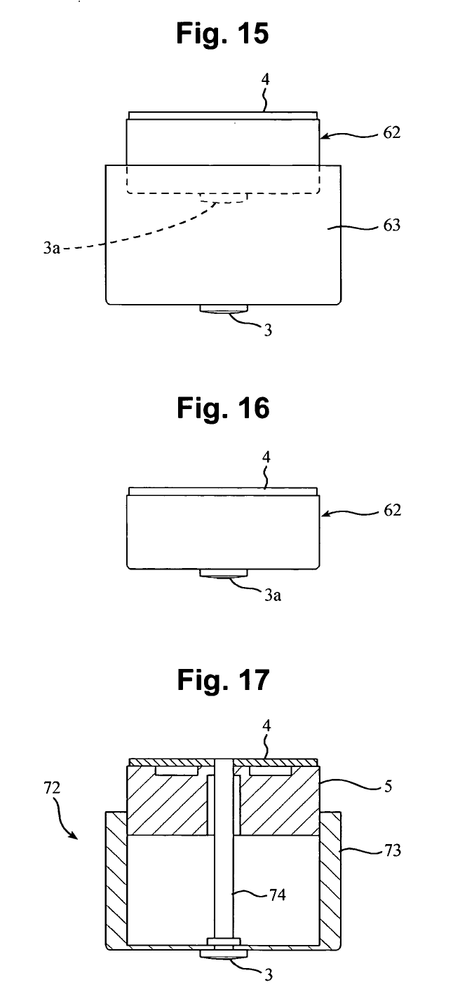

With such a configuration, said negative electrode 4 acts as a common negative electrode with respect to the positive electrode 3 and the positive electrode 3 a, and in the state in which the deeper-part measurement part 63 is mounted, the electrostatic capacitance across said positive electrode 3 providing a larger electrode spacing and the negative electrode 4 as shown in FIG. 15 is measured to determine the moisture content and concrete filled-in quantity for the deeper part of the concrete during placement 10, while, in the state in which the deeper-part measurement part 63 is removed, the electrostatic capacitance across said positive electrode 3 providing a smaller electrode spacing and the negative electrode 4 as shown in FIG. 16 is measured to determine the moisture content and concrete filled-in quantity for the closer part of the concrete during placement 10. In addition, the defect detecting/degree-of-filling-in measuring apparatus 1D of the present embodiment 4 uses the same switching type circuitry as that as shown in FIG. 13.

According to the defect detecting/degree-of-filling-in measuring apparatus 1D of the present embodiment 4, the deeper-part measurement part 63 is removably provided as an attachment to the main body of the defect detecting/degree-of-filling-in measuring apparatus 1D for concrete during placement, thus a single defect detecting/degree-of-filling-in measuring apparatus 1D having a simple configuration is capable of selectively and simply carrying out moisture content measurement, concrete filled-in quantity measurement, and defect detection for the closer part of the concrete during placement 10, or moisture content measurement, concrete filled-in quantity measurement, and defect detection for the deeper part thereof through the switching action.

### Embodiment 5

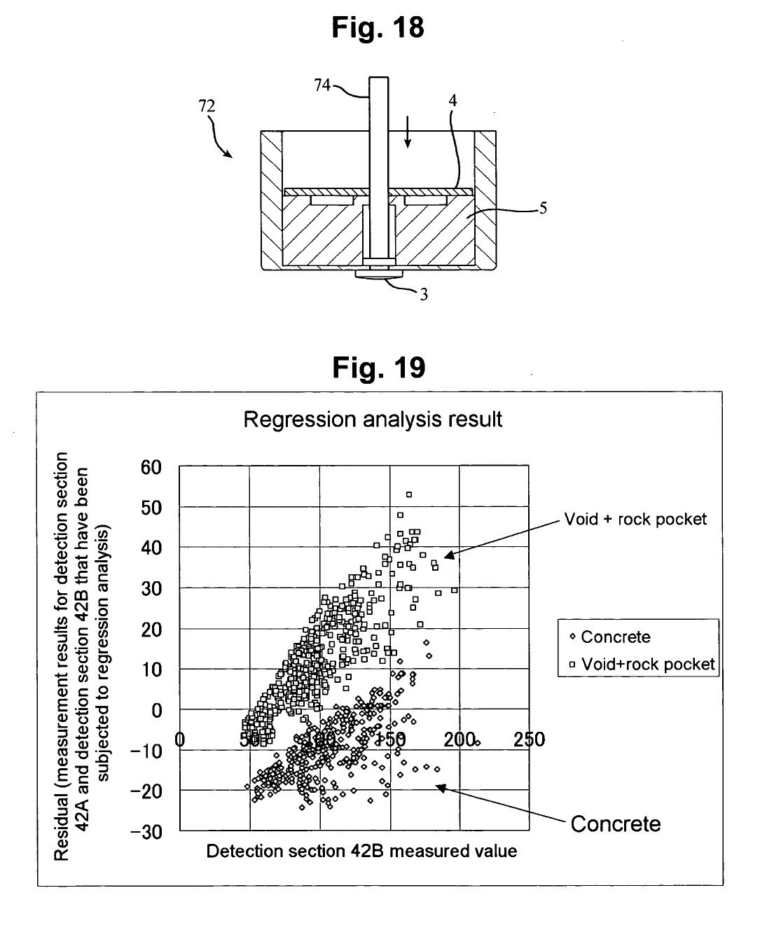

Next, with reference to FIG. 17 and FIG. 18, the electrode structure of a defect detecting/degree-of-filling-in measuring apparatus of an embodiment 5 of the present invention will be described. The present embodiment 5 features that the spacing between a positive electrode 3 and a negative electrode 4 of a detection section 72 is adjustable.

In other words, with the detection section 72 of the present embodiment 5, the positive electrode 3 is disposed in the middle portion of the end face of a cylindrical cover body 73 made up of an insulating material, and a guide body 74 is vertically provided in a perpendicular direction in the middle portion of the cylindrical cover body 73 such that the guide body 74 slidably supports the insulator 5 having the negative electrode 4 in a perpendicular direction.

According to the defect detecting/degree-of-filling-in measuring apparatus using the detection section 72 of the present embodiment 5, a single defect detecting/degree-of-filling-in measuring apparatus is capable of selectively carrying out determination of the moisture content and concrete filled-in quantity for the deeper part of the concrete during placement 10 to detect a defect with the negative electrode 4 being slid upward as shown in FIG. 17 to increase the electrode spacing between said positive electrode 3 and the negative electrode 4, or determination of the moisture content and concrete filled-in quantity for the closer part of the concrete during placement 10 to detect a defect with the negative electrode 4 being slid downward as shown in FIG. 18 to decrease the electrode spacing between said positive electrode 3 and the negative electrode 4.

Next, a method for defect detection/degree of filling-in measurement for concrete during placement 10 using any one of the defect detecting/degree-of-filling-in measuring apparatuses 1A to 1D and the apparatus having the configuration as shown in FIG. 17 and FIG. 18 as described above will be described.

This method for defect detection/degree of filling-in measurement is such that, in case where said defect detecting/degree-of-filling-in measuring apparatus 1A, for example, is used, the detection section 42A for deeper-part measurement and the detection section 42B for closer-part measurement are butted against the concrete form 9 simultaneously or alternately for measuring the electrostatic capacitance for the concrete; from the electrostatic capacitance value obtained using the detection section 42A for deeper-part measurement, the electrostatic capacitance value which is as a result of employing a predetermined approximate expression for correction of the electrostatic capacitance value obtained using the detection section 42B for closer-part measurement is subtracted to determine the moisture content and concrete filled-in quantity; and if the moisture content and the concrete filled-in quantity are low, as compared to the predetermined reference values, it is determined that the concrete during placement 10 has a filling-in defect. This is the same also with said defect detecting/degree-of-filling-in measuring apparatuses 1A′, 1B, 1C, 1D, and the defect detecting/degree-of-filling-in measuring apparatus having a configuration as shown in FIG. 17 and FIG. 18.

According to such a method for defect detection/degree of filling-in measurement, it becomes possible that, by using a single defect detecting/degree-of-filling-in measuring apparatus (any one of said defect detecting/degree-of-filling-in measuring apparatuses 1A to 1D, and the defect detecting/degree-of-filling-in measuring apparatus having a configuration as shown in FIG. 17 and FIG. 18), a filling-in defect in the concrete during placement 10 is easily detected.

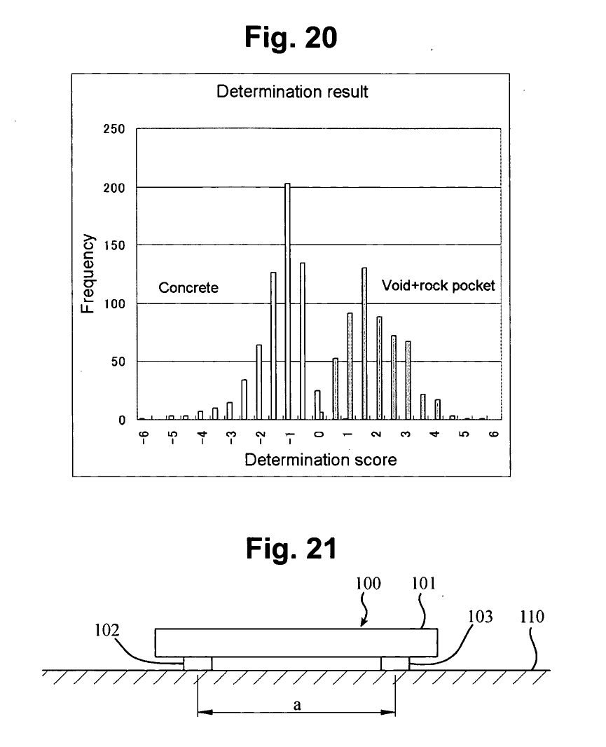

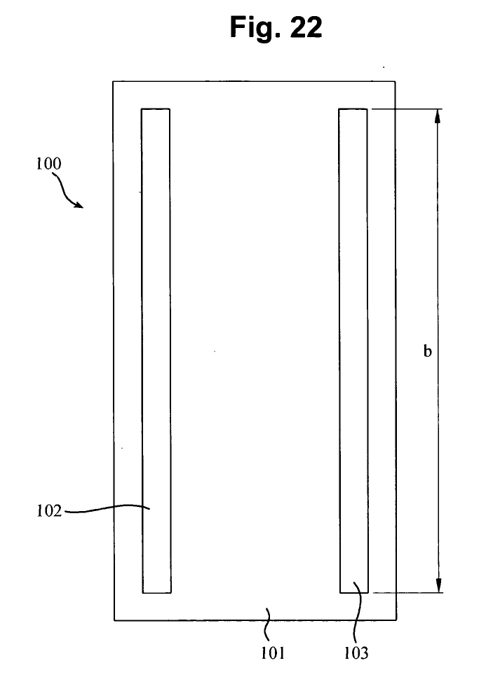

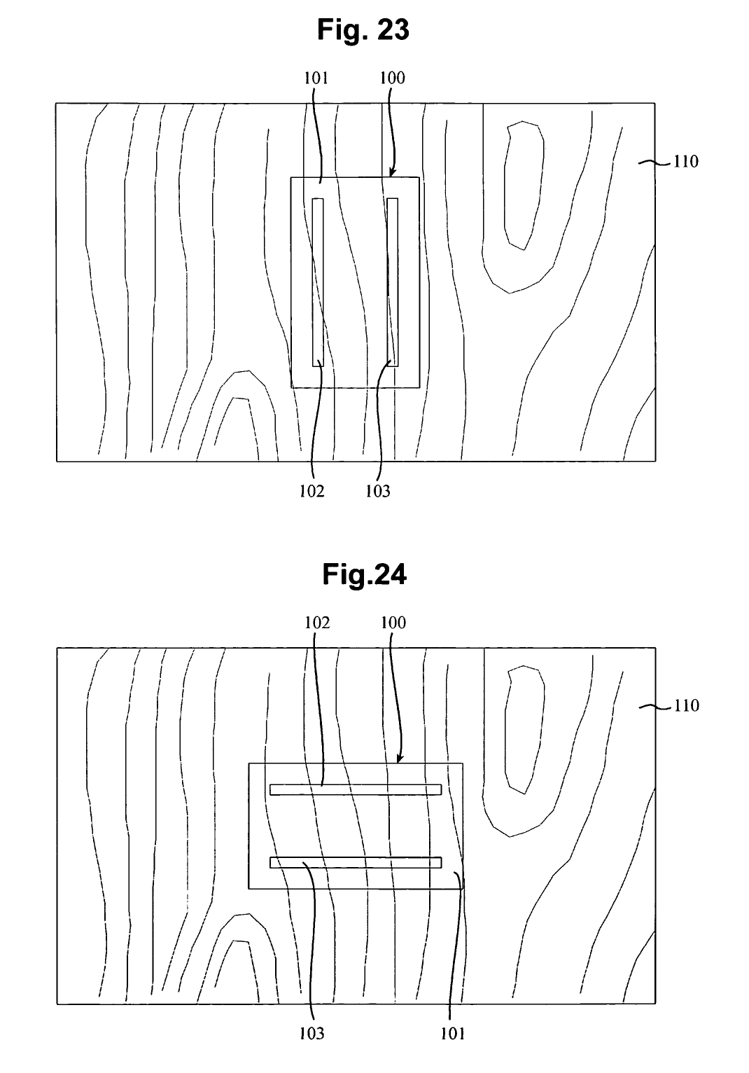

Next, with reference to FIG. 19 and FIG. 20, a specific example in case where said defect detecting/degree-of-filling-in measuring apparatus 1A, for example, is used to measure the concrete during placement 10 will be described. FIG. 19 is a regression analysis diagram about concrete and (void+rock pocket) that is given with the measured value for said detection section 42B being taken as the abscissa, and the residual (the measurement results for the detection section 42A and the detection section 42B that have been subjected to the regression analysis) being taken as the ordinate, and FIG. 20 is a bar graph illustrating the relationship between the determination score about concrete and (void+rock pocket) and the frequency, which has been prepared on the basis of a regression analysis result.

By obtaining such a regression analysis about concrete and (void+rock pocket) and a relationship between determination score and frequency, the condition of the concrete during placement can be exactly grasped.

### INDUSTRIAL APPLICABILITY

The present invention is widely applicable to moisture content measurement for grounds and wall surfaces in the field of various civil engineering works, concrete filled-in quantity measurement and defect detection, moisture content measurement for corn flour, etc., and the like, as well as defect detection/degree of filling-in measurement for concrete during placement as described above.

### Explanation of Reference Numerals Signs in the Drawings

1: Defect detecting/degree-of-filling-in measuring apparatus

1A: Defect detecting/degree-of-filling-in measuring apparatus

1A′: Defect detecting/degree-of-filling-in measuring apparatus

1B: Defect detecting/degree-of-filling-in measuring apparatus

1C: Defect detecting/degree-of-filling-in measuring apparatus

1D: Defect detecting/degree-of-filling-in measuring apparatus

2: Detection section

3: Positive electrode

3a: Positive electrode

4: Negative electrode

4a: Negative electrode

5: Insulator

5a: Circumferential edge part

6: Through-hole

9: Concrete form

10: Concrete during placement

11: Connection member

12: Lower connection receiving part

13: Upper connection receiving part

14: Connecting rod

15: Screw part

16: Flat washer

17: Spring washer

18: Nut

20: Gripper

21: Smaller diameter cylindrical part

22: Larger diameter cylindrical part

23: O-ring

25: Spacer part

26: Printed wiring board

27: Measuring circuit

28: Arithmetic circuit

29: Dry battery

30: Battery section

31: Display section

32: End member

33: Moisture content reference value memory (concrete filled-in quantity reference value memory)

41: Gripper

42A: Detection section

42B: Detection section

43: Deeper-part measuring circuit

44: Closer-part measuring circuit

45: Arithmetic circuit

45A: Arithmetic circuit

45a: Switching section

46: Display section

47: Moisture content reference value memory (concrete filled-in quantity reference value memory)

51: Gripping part

53A: Detection section holding part

53B: Detection section holding part

55: Detection section

61: Gripper

62: Closer-part measurement part

63: Deeper-part measurement part

72: Detection section

73: Cylindrical cover body

74: Guide body

1. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising:
a detection section having one electrode which surface to be butted against the concrete form is a curved surface; an insulator which is disposed such that a part of one end face thereof is adjacent to this one electrode; and the other electrode which is disposed at the other end face of said insulator, being opposed to said one electrode.

2. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, having:
a detection section having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode;
a measuring circuit which determines the electrostatic capacitance across said positive electrode and negative electrode from a detection signal of the detection section;
an arithmetic circuit which, on the basis of a measuring signal of the measuring circuit, determines whether the degree of filling-in for said concrete during placement is adequate; and
a display section which displays the result of operation of the arithmetic circuit.

3. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising:
a detection section for deeper-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode; and
a detection section for closer-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode, the spacing between said positive electrode and negative electrode being smaller than that for the detection section for deeper-part measurement;
at both ends of a gripper.

4. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising:
a detection section for deeper-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode; and
a detection section for closer-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode, the spacing between said positive electrode and negative electrode being smaller than that for the detection section for deeper-part measurement;
at both ends of a gripper,
and having:
a deeper-part measuring circuit which determines the electrostatic capacitance across said positive electrode and negative electrode from a detection signal of said detection section for deeper-part measurement;
a closer-part measuring circuit which determines the electrostatic capacitance across said positive electrode and negative electrode from a detection signal of said detection section for closer-part measurement;
an arithmetic circuit which processes the respective measured signals of said deeper-part measuring circuit and closer-part measuring circuit through the switching action to determine whether the degree of filling-in for said concrete during placement is adequate; and
a display section which displays the result of operation of the arithmetic circuit.

5. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising:
a detection section for deeper-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode; and
a detection section for closer-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode, the spacing between said positive electrode and negative electrode being smaller than that for the detection section for deeper-part measurement;
at both ends of a gripper which middle portion is bent at 90 degrees,
and having:
a deeper-part measuring circuit which determines the electrostatic capacitance across said positive electrode and negative electrode from a detection signal of said detection section for deeper-part measurement;
a closer-part measuring circuit which determines the electrostatic capacitance across said positive electrode and negative electrode from a detection signal of said detection section for closer-part measurement;
an arithmetic circuit which processes the respective measured signals of said deeper-part measuring circuit and closer-part measuring circuit through the switching action to determine whether the degree of filling-in for said concrete during placement is adequate; and
a display section which displays the result of operation of the arithmetic circuit.

6. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising:
a detection section for deeper-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode; and
a detection section for closer-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode, the spacing between said positive electrode and negative electrode being smaller than that for the detection section for deeper-part measurement;
as parts bifurcated from a gripper.

7. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising:
a detection section for deeper-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode; and
a detection section for closer-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode, the spacing between said positive electrode and negative electrode being smaller than that for the detection section for deeper-part measurement;
as parts bifurcated from a gripper,
and having:
a deeper-part measuring circuit which determines the electrostatic capacitance across said positive electrode and negative electrode from a detection signal of said detection section for deeper-part measurement;
a closer-part measuring circuit which determines the electrostatic capacitance across said positive electrode and negative electrode from a detection signal of said detection section for closer-part measurement;
an arithmetic circuit which processes the respective measured signals of said deeper-part measuring circuit and closer-part measuring circuit through the switching action to determine whether the degree of filling-in for said concrete during placement is adequate; and
a display section which displays the result of operation of the arithmetic circuit.

8. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising:
a detection section having a positive electrode which surface to be butted against the concrete form is a curved surface and which is disposed in the middle portion of one end face of a cylindrical insulator, and a negative electrode for deeper-part measurement which is disposed at the other end face of the cylindrical insulator, being opposed to said positive electrode; and
a negative electrode for closer-part measurement which is disposed in said insulator with a distance from the positive electrode that is smaller than that for said negative electrode.

9. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, having:
a detection section having a positive electrode which surface to be butted against the concrete form is a curved surface and which is disposed in the middle portion of one end face of a cylindrical insulator; a negative electrode for deeper-part measurement which is disposed at the other end face of the cylindrical insulator, being opposed to said positive electrode; and a negative electrode for closer-part measurement which is disposed in said insulator with a distance from the positive electrode that is smaller than that for said negative electrode;
a deeper-part measuring circuit which determines the electrostatic capacitance across said positive electrode and negative electrode for deeper-part measurement from a detection signal thereacross;
a closer-part measuring circuit which determines the electrostatic capacitance across said positive electrode and negative electrode for closer-part measurement from a detection signal thereacross;
an arithmetic circuit which processes the respective measured signals of said deeper-part measuring circuit and closer-part measuring circuit through the switching action to determine whether the degree of filling-in for said concrete during placement is adequate; and
a display section which displays the result of operation of the arithmetic circuit.

10. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising:
a gripper comprising a detection section for closer-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode;
a detection section for deeper-part measurement which is removably disposed with respect to said detection section for closer-part measurement, having a positive electrode which surface to be butted against the concrete form is a curved surface and which is disposed, being opposed to said negative electrode in the state in which it is mounted.

11. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising:
a gripper comprising a detection section for closer-part measurement having a positive electrode which surface to be butted against the concrete form is a curved surface and which is disposed in the middle portion of one end face of a cylindrical insulator, and a negative electrode which is disposed at the other end face of the cylindrical insulator, being opposed to said positive electrode;
a detection section for deeper-part measurement which is removably disposed with respect to said detection section for closer-part measurement, having a positive electrode which surface to be butted against the concrete form is a curved surface and which is disposed, being opposed to said negative electrode in the mounted state;
and having:
a deeper-part measuring circuit which determines the electrostatic capacitance across said positive electrode and negative electrode for deeper-part measurement from a detection signal thereacross in the state in which said detection section for deeper-part measurement is mounted;
a closer-part measuring circuit which determines the electrostatic capacitance across said positive electrode and negative electrode for closer-part measurement from a detection signal thereacross in the state in which said detection section for deeper-part measurement is removed;
an arithmetic circuit which processes the respective measured signals of said deeper-part measuring circuit and closer-part measuring circuit through the switching action to determine whether the degree of filling-in for said concrete during placement is adequate; and
a display section which displays the result of operation of the arithmetic circuit.

12. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, comprising:
a detection section having a positive electrode which surface to be butted against the concrete form is a curved surface; a cylindrical insulator which is disposed such that the spacing between it and the positive electrode is adjustable; and a negative electrode which is opposed to said positive electrode.

13. A defect detecting/degree-of-filling-in measuring apparatus for concrete during placement that measures the electrostatic capacitance for the concrete during placement from the outside of a concrete form for detecting a defect of and measuring the degree of filling-in for the concrete during placement, having:
a detection section having a positive electrode which surface to be butted against the concrete form is a curved surface; a cylindrical insulator which is disposed such that the spacing between it and the positive electrode is adjustable; and a negative electrode which is opposed to said positive electrode;
a deeper-part measuring circuit which determines the electrostatic capacitance across said positive electrode and negative electrode corresponding to the deeper-part measurement for the concrete during placement from a detection signal of said detection section when the spacing between said positive electrode and negative electrode is large, and which determines the electrostatic capacitance across said positive electrode and negative electrode corresponding to the closer-part measurement for the concrete during placement from a detection signal of said detection section when the spacing between said positive electrode and negative electrode is small;
an arithmetic circuit which, on the basis of the measurement signals of the measuring circuit, determines whether the respective degrees of filling-in for the deeper part and the closer part of said concrete during placement are adequate; and
a display section which displays the result of operation of the arithmetic circuit.

14. A defect detection/degree of filling-in measurement method for concrete during placement that uses any one of the defect detecting/degree-of-filling-in measuring apparatuses for concrete during placement as stated in claim 3, wherein
the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect.

15. A defect detection/degree of filling-in measurement method for concrete during placement that uses any one of the defect detecting/degree-of-filling-in measuring apparatuses for concrete during placement as stated in claim 4, wherein
the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect.

16. A defect detection/degree of filling-in measurement method for concrete during placement that uses any one of the defect detecting/degree-of-filling-in measuring apparatuses for concrete during placement as stated in claim 5, wherein
the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect.

17. A defect detection/degree of filling-in measurement method for concrete during placement that uses any one of the defect detecting/degree-of-filling-in measuring apparatuses for concrete during placement as stated in claim 6, wherein
the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect.

18. A defect detection/degree of filling-in measurement method for concrete during placement that uses any one of the defect detecting/degree-of-filling-in measuring apparatuses for concrete during placement as stated in claim 7, wherein
the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect.

19. A defect detection/degree of filling-in measurement method for concrete during placement that uses any one of the defect detecting/degree-of-filling-in measuring apparatuses for concrete during placement as stated in claim 8, wherein
the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect.

20. A defect detection/degree of filling-in measurement method for concrete during placement that uses any one of the defect detecting/degree-of-filling-in measuring apparatuses for concrete during placement as stated in claim 9, wherein
the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect.

21. A defect detection/degree of filling-in measurement method for concrete during placement that uses any one of the defect detecting/degree-of-filling-in measuring apparatuses for concrete during placement as stated in claim 10, wherein
the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect.

22. A defect detection/degree of filling-in measurement method for concrete during placement that uses any one of the defect detecting/degree-of-filling-in measuring apparatuses for concrete during placement as stated in claim 11, wherein
the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect.

23. A defect detection/degree of filling-in measurement method for concrete during placement that uses any one of the defect detecting/degree-of-filling-in measuring apparatuses for concrete during placement as stated in claim 12, wherein
the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect.

24. A defect detection/degree of filling-in measurement method for concrete during placement that uses any one of the defect detecting/degree-of-filling-in measuring apparatuses for concrete during placement as stated in claim 13, wherein
the detection section for deeper-part measurement and the detection section for closer-part measurement are butted against the concrete form simultaneously or alternately for measuring the electrostatic capacitance for the concrete; from the electrostatic capacitance value obtained using the detection section for deeper-part measurement, the electrostatic capacitance value obtained using the detection section for closer-part measurement is subtracted; and when the electrostatic capacitance value obtained by the subtraction is low, as compared to the predetermined reference value, it is determined that the concrete during placement has a filling-in defect.
